(19) United States
(12) Patent Application Publication
(10) Pub. No.: US 2011/0251659 A1
(43) Pub. Date: Oct. 13, 2011

(54) Method of Managing Metabolic Syndrome

(76) Inventor: Marvin Prescott, Los Angeles, CA (US)

(21) Appl. No.: 13/079,627

(22) Filed: Apr. 4, 2011

Related U.S. Application Data
(60) Provisional application No. 61/322,748, filed on Apr. 9, 2010.

(51) Int. Cl. A61N 5/06; A61N 5/067

(52) U.S. Cl. 607/89; 607/90

(57) ABSTRACT

A method is provided for treating metabolic syndrome with the use of a portable, non-invasive light-generating device. A laser beam is directed onto adipose tissue to reduce glucose and triglyceride levels through the activation of adiponectin. The laser beam is directed in accordance with a light regimen that is prescribed by a healthcare provide based one or more measured metabolic syndrome parameters of the patient.

### CROSS REFERENCE TO RELATED APPLICATIONS

This application claims priority to U.S. Provisional Patent Application Ser. No. 61/322,748, filed Apr. 9, 2010, and entitled “Metabolic Syndrome Management Device,” the entire disclosure of which is hereby incorporated by reference.

### FIELD

The present description relates generally to a method of using a light-generating device to direct a low power laser beam, according to a prescribed regimen, to specified areas of a patient's body for the treatment of metabolic syndrome.

### BACKGROUND/SUMMARY

Metabolic syndrome is a combination of medical disorders that may increase the risk of developing cardiovascular disease and diabetes. Some studies estimate that approximately 52 million individuals in the United States are afflicted with Metabolic Syndrome. Patients tend to have dyslipidemia (elevated triglycerides and reduced HDL cholesterol), hypertension, and high BMI. There is also a tendency for patients to develop visceral fat, leading to increased central obesity and elevated waist circumference.

Scientific research into the mechanisms of metabolic and cardiovascular diseases, such as metabolic syndrome and Diabetes, suggest that the protein hormone Adiponectin modulates a number of metabolic processes, including glucose regulation and fatty acid catabolism. Recent studies on cellular effects of Adiponectin and its role in human lipid metabolism and atherosclerosis suggest that adiponectin is an important autocrine/paracrine factor in the adipose tissue that modulates differentiation of preadipocytes and favors the formation of mature adipocytes. Further, Adiponectin can also function as an endocrine factor, influencing whole-body metabolism via effects on target organs. Research indicates that Adiponectin multimers exert differential biological effects, with high molecular weight multimers being associated with favorable metabolic effects (e.g., greater insulin sensitivity, reduced visceral adipose mass, reduced plasma triglycerides, and increased HDL-cholesterol). Likewise, Adiponectin appears to influence plasma lipoprotein levels by altering the levels and activity of key enzymes (such as lipoprotein lipase and hepatic lipase) responsible for the catabolism of triglyceride-rich lipoproteins and HDL (see Am. J. Clin. Nutr. (2010) January; 91(1):258S-261S, and Curr. Opin. Lipidol. (2007) June; 18(3):263-70). The pleiotropic effects of Adiponectin on adipose tissue homeostatis and the pathogenesis of metabolic syndrome, type 2 diabetes, and atherosclerosis, make it an attractive target for obesity-related therapies.

In a recent study on the role of Adiponectin in endothelial function, it was found that a loss of adiponectin in mice induced a primary state of endothelial dysfunction with increased leukocyte-endothelium (see J. Clin. Invest. (2007) June; 117(6): 1819-26). Specifically, prior administration of globular adiponectin (gAd) protected WT mice against TNF-α-induced leukocyte-endothelium interactions, indicating a pharmacologic action of gAd. Blockade of eNOS with L-NAME abolished the inhibitory effect of gAd on leukocyte adhesion, demonstrating the involvement of eNOS signaling in the anti-inflammatory action of gAd. It is now believed that gAd may protect vasculature in vivo by increasing the bioavailability of NO and suppressing leukocyte-endothelium interactions.

Current methods for managing metabolic syndrome involve invasive medical procedures, medication, and the use of large and complicated devices that are operated by healthcare professionals. Laser beam energy has been used in various medical and dental applications, such as pain control, wound healing, nerve stimulation, reduction of inflammation, arthritis, and stimulation of neuro-hormone systems. Novel potential medical applications of low power laser technology continue to be investigated. In a study conducted by Ignarro et al. (at UCLA, Los Angeles), porcine pedicle flaps were exposed to low power laser (at 850 nm) for selected durations following which changes in local and core temperature, blood flow, and vascular resistance were measured. A comparison of the measured parameters, as estimated in the presence and absence of L-NAME (a known Nitric Oxide Synthase (NOS) inhibitor) suggested that low power laser therapy may increase blood flow via a Nitric Oxide (NO) dependent mechanism, rather than a gross thermal effect. Since Adiponectin may affect lipid metabolism via NOS signaling, a method of treating metabolic disorders such as metabolic syndrome with a low power laser therapy directed at adiponectin has been developed. The method uses photons to activate the chromophore NOS. The resultant increase in NO levels may activate Adiponectin synthesis. Higher Adiponectin levels may, in turn, stabilize blood glucose levels, stimulate endothelial progenitor stem cells, and reduce endothelial dysfunction. By using laser beam energy from a portable light-generating device, metabolic syndrome may be managed by patients while going about their normal routines and without affecting their quality of life.

A method of managing metabolic syndrome using a portable light generating device is provided. The device can be attached to the skin over a stomach area of a patient's body, or other target areas of the patient's body that have underlying adipose tissue. A low power light beam generated by the device is directed to the underlying adipose tissue according to a light regimen prescribed by the patient's healthcare provider. The regimen may be adjusted based on one or more measured metabolic syndrome parameters of the patient, such as triglyceride levels, glucose levels, HDL levels, etc. The light regimen may include a plurality of emission periods (during which the light beam is directed towards the target area) separated by interval periods (during which no light beam is generated). The device may be worn until the measured parameters are within a desired range.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 shows a light-generating device that may be used to manage metabolic syndrome of a patient.

FIG. 2 shows an example method of treating metabolic syndrome in a patient using the light-generating device.

FIGS. 3-4 show example embodiments wherein metabolic syndrome is managed by attaching the light-generating device to a visceral adipose area of the patient's body.

FIG. 5 shows an example embodiment wherein metabolic syndrome is managed by attaching the light-generating device to a thigh area of the patient's body.

### DETAILED DESCRIPTION

Conventional low power laser therapeutic devices generally comprise a hand-held probe with a single laser beam source, or a large, stationary, table console with attached probe(s) powered by a conventional fixed power supply. Such large, multi-beam devices are typically very expensive and require extensive involvement of medical personnel when treating a patient. For example, the device may be affixed to a stand which has to be focused and controlled by a doctor or ancillary medical personnel. In addition to adding to the cost of the device and the treatment therewith, such a device requires a patient to travel to the location of the laser treatment device in order to obtain the laser therapy. Studies have shown that such treatment typically must be provided on a regular basis (e.g., every few hours or daily) once the treatment is initiated in order to be effective and to produce optimum results. This requires numerous patient visits to the treatment facility or extensive waiting on the part of the patient. In addition to increasing the patient's financial costs, the frequent visits reduce the patient's quality of life. For example, in addition to being away from family members, the patient is generally incapable of working or otherwise being productive while at the treatment facility. On the other hand, if the patient is inconsistent about the scheduled visits, the efficiency of the treatment regimen is reduced and the patient's health risks can increase.

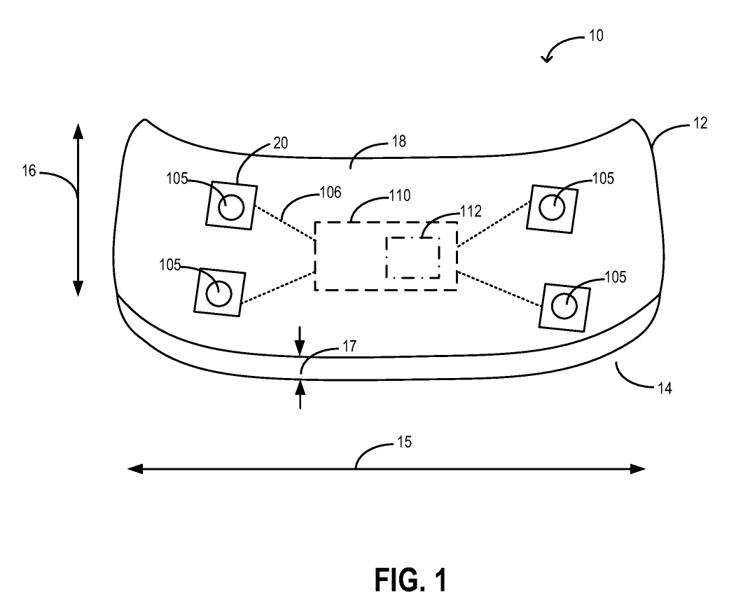

To address these issues, while enabling a person suffering from metabolic syndrome to use laser therapy in the management of metabolic syndrome, the method of managing metabolic syndrome described herein uses a portable light-generating device. An example embodiment of such a device is shown in FIG. 1. One or more light-generating devices 10 may be attached on the skin of a patient's body, for example, to a specific body part or body region, and operated according to a prescribed light regimen. The specified body part or region may be regions that have underlying adipose tissue, such as stomach (visceral) areas, thigh areas, and upper leg areas. The prescribed light regimen may be determined by the patient's healthcare provider based on one or more metabolic syndrome parameters of the patient. For example, the light regimen may be based on the patient's triglyceride levels, glucose levels, cholesterol levels, lipoprotein levels and/or ratios (e.g., HDL level, or HDL to LDL ratio), etc. The light regimen may include a plurality of emission periods during which low power light is directed towards the specific body part. Each of the plurality of emission periods may be separated from each other by interval periods during which no low power light is generated. As such, the light regimen may be continued until the measured parameters are within or below a threshold range. Example light regimens are discussed below.

Light-generating device 10 may have a body 12 that is made of p-cell or plastazote or lux plastic to make it non-flexible, light-weight, and portable. In an alternate embodiment, the body of the light-generating device may be made of flexible materials, such as molded silicone, to render the device is flexible, light-weight, and portable. The body 12 may be designed as a casing within which are housed a light source, a power source, a programmable controller, and other sensors of the device 10. The device may be designed as virtually any shape. As a non-limiting example, the device 10 is depicted with a substantially rectangular body 12 having a length 5 inches, a width 2.5 inches, and a thickness ¼ inch. The dimensions of the device may be adjusted based on the desired application of the device. As a non-limiting example, when used for the management of metabolic syndrome, the dimensions of the body may include an average length of 5 inches, an average width of 2.5 inches and an average thickness of ¼ inch. The body 12 may be contoured to reduce discomfort when the device is attached to the patient's body. The contour 14 of the device may be adjusted to conform to the body part to which it is to be attached. For example, the device may be contoured differently if designed for use on the stomach or visceral adipose area than when designed for use on the thigh or upper leg area.

The body 12 of the device 10 may include openings 20, formed therein, to accommodate light produced by an underlying light source 105. The openings 20 may be provided with clear windows to protect the underlying light source from dust, oils, dirt, etc. The clear windows may be formed of glass, plastic, or other suitable materials. Alternatively, the clear windows may be formed of the same material as the body of the device. As such, this would allow the windows to be co-molded with the body 12.

In some embodiments, device 10 may be encased in a pre-formed, sterilized, disposable cover (not shown) held in place by a friction fit over body 12. The cover may further include a cushioning agent to cushion the device when attached to the patient's body. The disposable or sterilized nature of the cover ensures that only sterile surfaces contact the patient's body and treatment area. When included, the cover may also have a plurality of openings that are aligned with the (underlying) openings 20 of body 12. In some embodiments, body 12 may further include strap hooks (not shown) for receiving a strap for supporting the device. In other embodiments, the device may be attached to a target area of the patient's body using flexible bandage.

In the depicted example, device 10 includes four light sources 105 distributed over the surface area of the device. The light sources 105 may be configured to generate a low power light. As such, the light sources may be configured to be flexible or non-flexible, based on the flexible nature of the device. As non-limiting examples, the light sources may include any combination of lasers (or laser diodes), light emitting diodes (LEDs), and/or infrared devices. Based on the nature of the light source, the light beam generated by the device may have a wavelength ranging from 400 nm to 1300 nm.

In some embodiments, device 10 may further include a focusing lens (not shown) for focusing the low power light generated by the light source onto the body of the patient. However in alternate embodiments, such as where the light source is capable of generating a collimated and focused beam, additional focusing lenses may not be required.

Light sources 105 may be interconnected inside the device 10 by flat wire connectors 106 (depicted with dotted lines). The flat wire connectors 106 may lead to a power source 110 and a programmable controller 112. Power source 110 powers light source 105. Various small and portable power sources can be used, such as one or more batteries. The batteries may include, for example, one or more wafer thin lithium polymer batteries, or rechargeable nickel-metal hydride batteries. In one example, when rechargeable batteries are included in power source 110, the batteries may be recharged when device 10 is not enabled, or not attached to the patient's body. Programmable controller 112 may include a control circuit printed onto a circuit board. As such, programmable controller may be configured with code for selectively enabling the light source 105 of the device 10 according to the prescribed light regimen. Specifically, the programmable controller may selectively enable the light source (for example, by connecting light source 105 to power source 110) during each of the emission periods of the prescribed light regimen, and then selectively disable the light source (for example, by disconnecting light source 105 from power source 110) during each of the interval periods of the prescribed light regimen.

Device 10 may optionally include a proximity sensor (not shown) for determining the proximity of the device to the patient's body. The proximity sensor may be configured as a chip included in the body 12 of the device. In one example, the proximity sensor may be activated when the device 10 is within a threshold distance of the patient's body (e.g., when the device is placed on the waist/visceral area of the patient). The proximity sensor may communicate that the sensor (or the device) is within a threshold distance of the patient's body to the healthcare provider (e.g., via wireless communication). In this way, with the help of the proximity sensor, a doctor may be able to monitor the wearing of the device and make sure that the patient is wearing the device as prescribed.

The device may further include one or more parameter sensors (not shown) for measuring a metabolic syndrome parameter of the patient. These may include, for example, glucose levels, cholesterol levels, HDL levels, blood pressure, blood oxygen levels, etc. Based on the parameters to be measured, appropriate parameter sensors may be included in the device.

Since the body 12 of the device houses the light sources, power sources, programmable controller, and various sensors, support posts may be optionally included within body 12 to enhance the rigidity of the device, as well as to provide spacing within the body to delineate the internal components.

In one embodiment (as depicted), light sources 105 are low-power lasers, such as vertical-cavity surface-emitting lasers (VCSELs) having a nominal power output of 2.6 mW and a wavelength in the range of 600-850) nm (herein, a wavelength of approximately 780 nm). The power output of the lasers may range from 2.6 to 10 mW. At this operating level, sufficient power can be provided for the treatment of metabolic syndrome with an extended life of the power source.

As such, VCSELs comprise semiconductor lasers which emit a collimated beam normal to the surface of the semiconductor substrate. The semiconductor typically comprises aluminum arsenide (AlAs) or gallium arsenide (GaAs), or a combination thereof. Each VCSEL has a self contained, high-reflectivity mirror structure forming a cavity to produce the collimated beam. Due to the ability of VCSELs to produce a collimated beam, additional focusing lenses may not be required in device 10 to focus the beam when VCSELs are the light source. Additionally, VCSELs provide reduced size and low power consumption benefits. Since typical VCSELs are in the order of 25 micrometers long, and have an operational power threshold below 1 milliamp, multiple VCSELs can be powered from a single power source.

In one embodiment, the VCSELs are one millimeter or less in height and are embedded in the casing material of body 12. Arrays of VCSELs, spaced by one-half inch, may be “sandwiched” between the polymer material and interconnected by flat wire connectors 106 in the device. The arrays of VCSELs may be electrically connected in parallel to avoid having a single inoperative VCSEL prevent numerous other VCSELs from operating.

In another embodiment, the light source includes one or more light emitting diodes (LEDs), such as hyper-luminescent red (“hyper-red”) LEDs. These LEDs can emit a light beam having 2000-3000 candle power and a wavelength of approximately 670 nanometers. The hyper-red LEDs may also be arrayed in a manner similar to the VCSELs in device 10. In still other embodiments, a combination of LEDs and VCSELs may be used wherein the hyper-red LEDs and the VCSELs are arrayed in an alternating fashion with several VCSELs for every hyper-red LED.

Operation of the power source 110 and programmable controller 112 may be initiated by means of a switch, such as a single-pole, double-throw or pressure switch. The programmable controller 112 may be configured with code for controlling the operation and timing of light sources 105. As used herein, timing control includes enabling the operation of the light source for the duration of the emission period, and disabling the operation of the light source for the duration of the interval period, in accordance with the prescribed light regimen. The programmable controller may have a 24 hour timing function which adjusts the operation of the light source for the emission and interval periods of time over the course of the 24 hour period.

The programmable controller may further include an oscillator to supply pulses at regular intervals (e.g., at one second intervals) to a counter/timer circuit. The counter circuit counts the pulses while a count decode logic circuit monitors the count. The count decode logic circuit is a multipurpose logic circuit which may comprise, for example, a PAL (programmable array logic) or a PLA (programmable logic array) that may be programmed to detect specified counts based on the emission periods and interval periods of the light regimen. For example, a count of 14,400 which would correspond to four hours of time and a count of 120 which would correspond to two minutes of time. Programmable controller 112 would be capable of maintaining the stored timing program without power being applied thereto. Upon detecting the programmed count, the count decode logic circuit outputs a laser enable pulse which enables laser current regulator circuits which regulate the power to each laser or LED of the light source. The regulator circuits, which are known in the art and which compare the current with a known voltage reference in order to maintain a constant current output, receive a voltage reference input from a voltage reference circuit. The voltage reference circuit may comprise an active bandgap zener diode which supplies a constant voltage output (on the order of 1.2 to 1.5 volts) regardless of the voltage of the battery. At the same time, the logic circuit provides a RESET pulse to the counter/timer circuit to reset the count, and the counter continues counting the pulses from the oscillator. The laser enable pulse remains active for the duration of the emission period of the light regimen.

When power source 110 includes a rechargeable battery, the programmable controller 112 may also be configured to connect power source 110 to an external power source to recharge the battery. In some embodiments, power source 110 may be further coupled to a dedicated battery charge controller that is configured to monitor charging of the rechargeable battery and disconnect the battery from the external power source when a threshold charge level is reached.

Power source 110 may be selected so that it is capable of providing sufficient power for a defined duration of the laser therapy (e.g., one day, one week, etc.). A low battery voltage protection circuit may be included in programmable controller 112 to regulate the power supplied by the power source 110 and provide a desired voltage output (e.g., between 3.6 and 4.8 volts). The protection circuit can cease the supply of power if the battery voltage drops below the threshold level (e.g., 3.6 volts) to avoid damage to circuit components. The power supplied by the power source 110 via the protection circuit is used to power the circuit components as well as the VCSELs and/or hyper-red LEDs. A standard LED driver circuit, as known in the art, may be used to drive the hyper-red LEDs.

In some embodiments, device 10 may include a heat sink coupled to each light source to address excess heat generated by the light source during device operation. The heat sinks may be formed of copper and can be used to disburse the excess heat produced by the laser diode light source into the surrounding housing material. The heat sinks may also lend added structural integrity to the locations of the laser diodes. However, when VCSELs are used as the light source, the heat sinks may not be required due to low heat generated by the VCSELs.

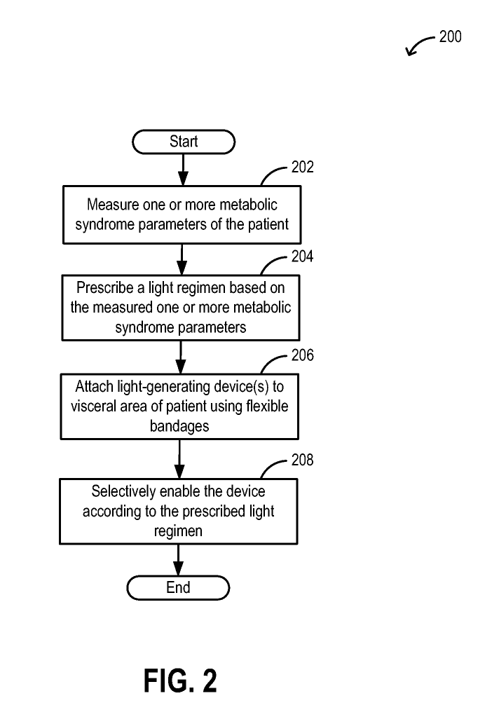

Now turning to FIG. 2, an example method 200 of managing metabolic syndrome with a portable light generating device, such as the device of FIG. 1, is provided. At 202, the method includes measuring one or more metabolic syndrome parameters of the patient. For example, a blood glucose level may be estimated. At 204, the method includes prescribing a light regimen based on the measured metabolic syndrome parameter. Specifically, the healthcare provider may program the programmable controller with the details of the prescribed light regimen. In one example, the programmable controller may be preprogrammed with a plurality of light regimens from which the healthcare provider may select an appropriate light regimen depending on the patient's measured metabolic syndrome parameter. In an alternate example, the programmable controller may be provided with a PCMCIA port which can interface with a “smart card” or master programming card which can be inserted to download a desired prescribed light regimen on to the controller by the healthcare provider. As still another example, a serial interface may be provided to connect the light-generating device to a personal computer of the healthcare provider, from where the prescribed light regimen may be downloaded on to the controller.

At 206, the method includes attaching the light-generating device to a target area (e.g., stomach area, thigh area, etc.) of the patient's body. In the depicted example, the device is attached to a visceral adipose area of the body (that is, a visceral area with underlying adipose tissue). The light-generating device may be attached to the selected part of the patient's body by the healthcare provider (e.g., physician) using a strap or bandage, so as to position the device over the desired treatment location. The strap or bandage may be sterilized, disposable, and made of a stretchable or flexible material (such as nylon) to allow the device to be properly attached to the patient's body while applying a positive biasing force to hold the device in place. Once placed upon the patient's body, the device may essentially look like a “patch”. This improves the wearability of the device. The patch can be worn by the patient for 12 hours per day, or 12 hours at night, and recharged when not worn. Further, since different light regimens can be provided for the patient using the same device (by changing the details on the programmable controller), patients may not need to replace the device frequently. For example, the patient may need a new patch only once every two years.

At 208, the method includes selectively enabling the device according to the prescribed light regimen. As such, operation of the device may be initiated by operating a switch. The light sources of the device may be configured to provide a visible feedback to the user (e.g., by flashing once) to confirm that the device is operational. The healthcare provider may operate the switch upon attaching the device to the patient's body. After the switch is operated and the device is attached to the desired area, the prescribed light regimen can begin.

Optionally, a lag period may be provided between the operation of the switch and the actual initiation of the light regimen to allow sufficient time for the device to be properly positioned on the patient's body prior to initiation of the laser therapy. Alternatively, the switch may be activated upon the prior activation of the proximity sensor, ensuring that the light regimen is initiated only when the device is placed on the patient's body.

After being located on the patient's body, the patient simply wears the device for the prescribed period of time and is free to conduct themselves in the normal course. The device automatically delivers the laser therapy according to the prescribed light regimen, as indicated in the programmable controller, while the patient goes about their normal routine. With such a device, a single doctor visit replaces time consuming, and costly, multiple office visits. A physician can initiate the laser treatment regimen using the light-generating device and allow the patient to leave with the device attached to their body for a defined duration, such as one week of laser therapy, without the need for the patient to return to the physician's office. An appointment can be scheduled after that duration for the physician to determine the success of the laser therapy and adjust (or reprogram) the light regimen, for example, based on newly measured metabolic syndrome parameters of the patient. As such, since the device provides precisely timed laser treatments to target areas, the efficiency of the laser treatment is optimized and an overall length of the required treatment can be reduced.

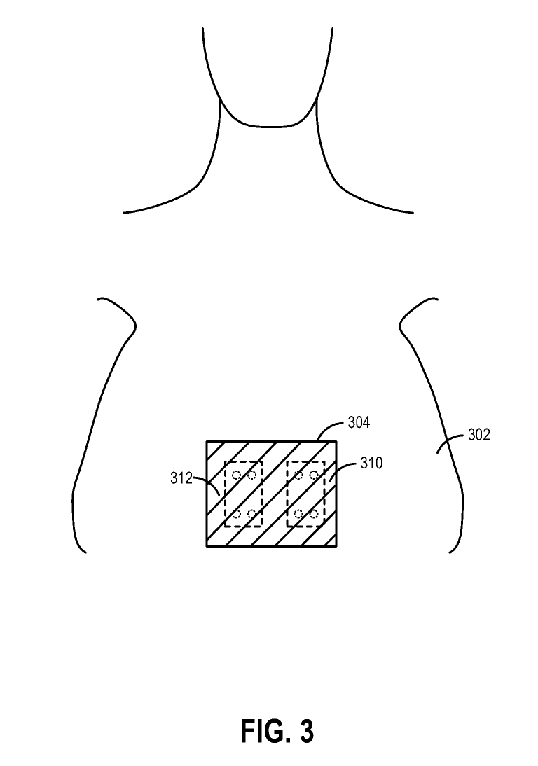

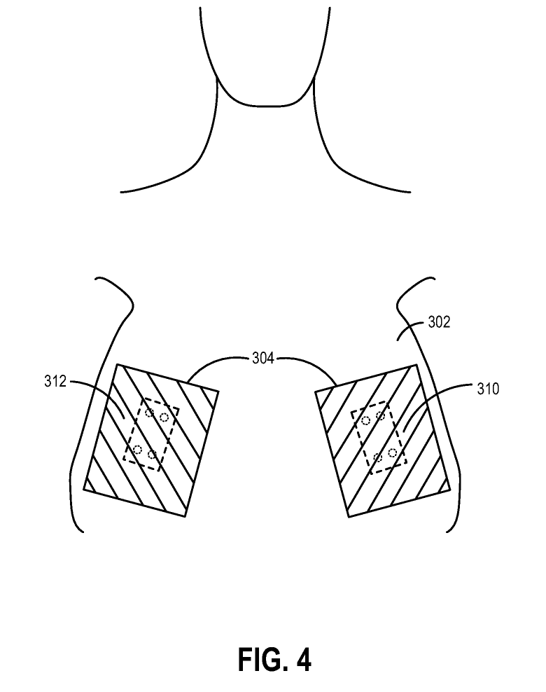

FIGS. 3 and 4 illustrate example embodiments of a method of managing metabolic syndrome wherein the device is attached to a stomach area 302 (or visceral adipose area) of the patient's body. Specifically, in FIG. 3, a plurality of light-generating devices 310, 312 (herein, two devices) are positioned along a waist circumference, specifically at the level of the navel, to target the low power laser light to the adipose tissue underlying the waist circumference. In the depicted example, each device 310, 312 includes four lasers (dotted circles). The devices 310, 312 are kept in place with bandage 304. In FIG. 4, the plurality of light-generating devices are arranged on the sides of the stomach area 302 area. Specifically, the plurality of devices are distributed into a left side device 310 that targets the low power laser light on the left side of the stomach area 302, and a right side device 312 that targets the low power laser light on the right side of the stomach area 302. The devices 310, 312 are kept in place with bandage 304. In both FIGS. 3-4, light beams from the light-generating devices are focused on the patient's visceral adipose tissue. Since the visceral adipose tissue is one of the regions where triglycerides and peptides are stored and released, by focusing the light beams on this target area, the user's sensitivity (e.g., glucose sensitivity) to the light therapy can be increased. As such, the adipose tissue emits triglycerides and causes endothelial dysfunction, which can lead to cardiovascular disease, kidney failure, and diabetic retinopathy. Thus, by causing an increase in glucose sensitivity, the laser therapy using the light-generating devices may be used to manage the patient's metabolic syndrome by reversing the blood chemistry of individuals with metabolic syndrome, pre-diabetic syndrome, or diabetes.

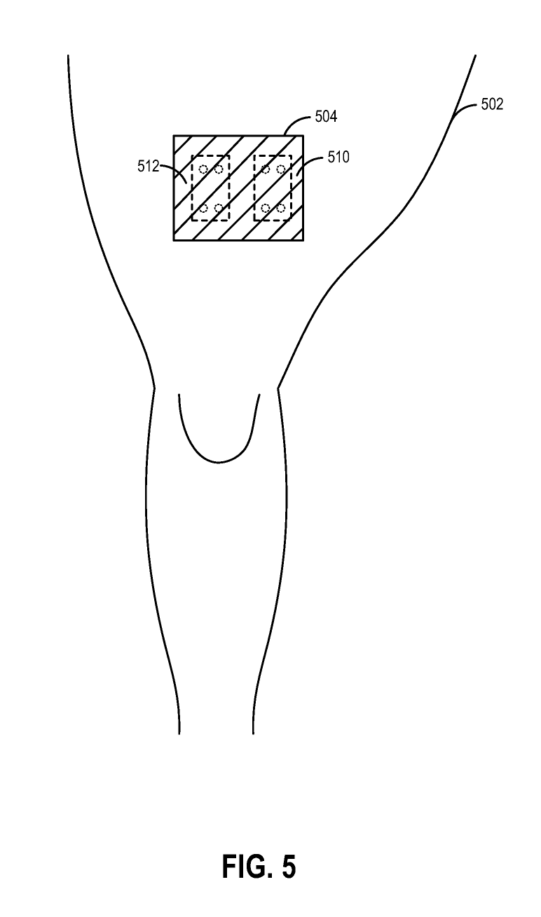

FIG. 5 illustrates another example embodiment of a method of managing metabolic syndrome. Specifically, in FIG. 5, the plurality of light-generating devices 510, 512 are positioned along a front portion of the patient's thigh area 502, using bandage 504, to target the low power laser light to the adipose tissue underlying the thigh and upper leg area. While FIG. 5 shows the plurality of devices aligned along a front portion of the thigh area, it will be appreciated that the devices may alternatively be positioned on left and right side thigh regions, or front and back regions of the thigh area, similar to the positioning shown in FIG. 4.

In one example light regimen, the patient may wear two light-generating devices, each device having 4 lasers as the light source, that emit light beams over the underlying adipose tissue of the user for 12-14 hours per day. In each device, two of the four lasers may be selectively enabled to dose 9-12 mW of light energy on the target area for an emission period of two minutes before being disabled. Next, the other two lasers of the device may be selectively enabled to dose 9-12 mW of light energy on the target area for an emission period of two minutes before being disabled. The lasers of the devices may be selectively disabled for an interval period of sixteen minutes. As such, the emission period followed by the interval period constitutes a cycle which is then repeated over the duration of the regimen. That is, at the end of the sixteen minutes, the lasers may be re-enabled for two minute durations. In one example, the patient may wear the device for 14 hours during which the cycle (with emission periods separated by interval periods) is constantly repeated. At the end of the 14 hours, the device may be recharged. In one example, where the power source of the device is rechargeable (e.g., lithium ion battery), the power source is recharged for 5 hours after the 14 hour regimen, each day.

A method of treating metabolic syndrome with the afore-mentioned light regimen was tested on patients with metabolic syndrome and type II diabetes for 18 months. In each patient, 12-hour blood glucose levels, triglyceride levels, oxidized LDL levels, and adiponectin levels were estimated before and after treatment with the prescribed light regimen, and/or with and without the prescribed light regimen. Also, patient weights were checked at 3 months and 6 months since the initiation of the treatment. Some of the results of the study are listed below in Tables I and II. As such, the results indicate that the light regimen led to normalized blood glucose levels and a reduction in triglyceride levels to within normal levels. Further, in cases where adiponectin levels were not within the normal range before treatment, within three months of wearing the device and receiving laser therapy according to the prescribed light regimen, adiponectin levels were within the normal range.

Table I shows example results from a first patient having Metabolic Syndrome. The patient is a 46-year old female. Patient was tested with a light-generating device having 8 lasers. Patient's diet was maintained, with no changes, over the duration of the treatment. Also, no diabetic drugs were administered. In addition to lowering of glucose and triglyceride levels, the original waist size of the patient was reduced by 2 inches in one month. Further, adiponectin was returned to a normal range within 4 months of treatment.

TABLE I

| | Pre-Laser | 4 weeks Post |
|---|---|---|
| Blood Test | Treatment | Laser-Treatment |
| Fasting Glucose | 103 | 94 |
| Cholesterol | 144 | 171 |
| Triglyceride | 215 | 203 |
| HDL | 32 | 35 |
| LDL calculated | 69 | 95 |
| Cholesterol/HDL | 4.5 | 4.9 |

Table II shows example results from a second patient having type II Diabetes. The patient is a 49 year old male. Patient was tested with a light-generating device having 8 lasers. The patient wore the device for 10 hours per day. Prior to treatment with any therapy, the patient's blood glucose level was 468. The patient was prescribed the diabetic drugs Metformin and Lantus to reduce glucose levels. The patient gained 35 lbs during the first month of treatment with Lantus. After starting treatment with the laser therapy, in addition to stabilizing glucose levels, the original waist size of the patient was reduced by 2.5 inches while weight was reduced by 32 lbs. The patient has been able to manage Diabetes without taking Lantus. The patient was also able to reduce Metformin usage by half. The patient's blood pressure was returned to within a normal range. Further, adiponectin was returned to a normal range (7 mcg/l) within 4 months of treatment.

TABLE II

| | Pre-Laser | 4 months Post | 21 months Post |
|---|---|---|---|
| Blood Test | Treatment | Laser- Treatment | Laser-Treatment |
| Glucose | 94 | 94 | 74-100 |
| Cholesterol | 144 | 171 | 171 |
| Triglyceride | 92 | 133 | 78 |
| HDL | 49 | 49 | 56 |
| Cholesterol/HDL | 2.3 | 2.7 | 2.2 |
| Lantus | 32 u | 10 u | 0 u |
| A1C | 5.2 | 5.8 | 5.7 |

In summary, results suggest that by using a portable laser device, metabolic syndrome of a patient may be managed. In particular, by attaching the laser device to a stomach area having underlying adipose tissue, and selectively enabling the laser device according to a prescribed light regimen, glucose levels and triglyceride levels may be reduced. By adjusting the prescribed light regimen based on measured metabolic syndrome parameters of the patient, such as glucose levels and triglyceride levels, the therapy can be customized for each patient based on the severity of their condition. By using a portable and non-invasive device to provide the laser therapy, the treatment can be undergone without adversely affecting the patient's quality of life.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof.

1. A method of using a light-generating device for managing metabolic syndrome in a patient, the device including a light source for generating low power light, a power source for powering the light source, a proximity sensor for determining a proximity of the device to the patient, a parameter sensor for measuring a metabolic syndrome parameter of the patient, and a programmable controller for selectively enabling the light source of the device according to a prescribed light regimen, the method comprising:
attaching the device to a body part of the patient, the body part having underlying adipose tissue; and
selectively enabling the device according to the prescribed light regimen to direct a beam of low power light from the device onto the body part of the patient, the prescribed light regimen based on the measured metabolic syndrome parameter of the patient.

2. The method of claim 1, wherein the body part includes a stomach visceral adipose area, a thigh area, and/or an upper leg area.

3. The method of claim 1, wherein the light source includes a vertical cavity surface emitting laser diode and/or a light emitting diode.

4. The method of claim 3, wherein the light source has a wavelength of 850 nm and a power of 9 to 12 mW.

5. The method of claim 1, wherein the power source includes a rechargeable battery, and wherein the device includes a focusing lens for focusing the low power light generated by the light source.

6. The method of claim 1, wherein the light regimen includes a plurality of emission periods during which the low power light is directed towards the body part, each of the plurality of emission periods separated from each other by an interval period during which no low power light is generated.

7. The method of claim 6, wherein each of the plurality of emission periods is two minutes in length, and wherein each interval period is between two and sixteen minutes length.

8. The method of claim 1, further comprising, communicating when the proximity sensor is within a threshold distance of the patient's body to a healthcare provider of the patient via wireless communication.

9. The method of claim 1, further comprising, continuing the prescribed light regimen until the metabolic syndrome parameter measured by the device is below a threshold.

10. A method of treating a patient, comprising:
measuring a metabolic syndrome parameter of the patient using a non-flexible light-generating device, the device including a plurality of low power lasers for generating a low power light beam, a rechargeable power source for powering the plurality of low power lasers, and a proximity sensor for communicating a distance between the sensor and the visceral area of the patient to a healthcare provider, the device contoured to match the visceral adipose area of the patient;
attaching the device to the visceral adipose area of the patient using a bandage; and
operating the device according to a light regimen to direct the low power light beam on the visceral area of the patient.

11. The method of claim 10, wherein the plurality of lasers are vertical cavity surface emitting laser diodes.

12. The method of claim 10, wherein the power source is recharged when the device is not attached to the patient.

13. The method of claim 10, further comprising, during operation of the device, communicating when the proximity sensor is within a threshold distance of the patient's body to the healthcare provider via wireless communication.

14. The method of claim 10, wherein the light regimen includes a plurality of emission periods separated from each other by an interval period, and wherein operating the device according to the light regimen includes directing the low power light beam on the visceral adipose area during each emission period, and not generating the low power light beam during each interval period.

15. The method of claim 10, further comprising, continuing operation of the device according to the light regimen until the metabolic syndrome parameter measured by the device is below a threshold.

16. A method of treating a patient having metabolic syndrome with a non-flexible light-generating device, the device including a low power laser that emanates a low power light beam directed towards a visceral area of the patient's body, a rechargeable power source for powering the low power laser, a proximity sensor for measuring a proximity of the device to the visceral area of the patient's body, the proximity sensor communicatively coupled to a communication device of a healthcare provider, and a parameter sensor for measuring one or more metabolic syndrome parameters of the patient, the device contoured to match the visceral area of the patient, the method comprising:
attaching the device to a visceral adipose area of the patient;
determining a light regimen of the device based on the one or more metabolic syndrome parameters of the patient measured by the device, the determined light regimen including a plurality of emission periods during which low power light is directed towards the visceral adipose area, each of the plurality of emission periods separated from each other by an interval period during which no low power light is generated; and
operating the device according to the light regimen to direct the low power light on to the visceral area of the patient, wherein the operating includes enabling the low power laser of the device during each of the plurality of emission periods, and disabling the low power laser of the device during each interval period.

17. The method of claim 16, wherein each of the plurality of emission periods has a duration of two minutes, and wherein each interval period has a duration of two to sixteen minutes.

18. The method of claim 16, wherein the laser has a wavelength of 850 nm and a power of 9 to 12 mW.

19. The method of claim 16, wherein the one or more metabolic syndrome parameters include blood glucose level, blood triglyceride level, blood cholesterol level and/or blood lipoprotein level.

20. The method of claim 16, wherein the device has a contour that matches a visceral region of a human body, and wherein the device is attached to the visceral area of the patient's body with one or more flexible bandages.
